(19) United States
(12) Patent Application Publication
(10) Pub. No.: US 2013/0052249 A1
(43) Pub. Date: Feb. 28, 2013

(54) ANTIMICROBIAL COMPOSITION

(71) Applicants: Pentti Sipponen, Espoo (FI); Kari Holopainen, Lahti (FI); Pasi Keinanen, Muurame (FI); Joachim Karthauser, Sollentuna (SE)

(72) Inventors: Pentti Sipponen, Espoo (FI); Kari Holopainen, Lahti (FI); Pasi Keinanen, Muurame (FI); Joachim Karthauser, Sollentuna (SE)

(73) Assignee: TAIGA POLYMERS OY, Helsinki (FI)

(21) Appl. No.: 13/500,234

(22) PCT Filed: Oct. 8, 2010
(86) PCT No.: PCT/FI10/50787

§ 371 Date: Apr. 4, 2012

(30) Foreign Application Priority Data
Oct. 8, 2009 (FI) 20090371
Oct. 8, 2009 (FI) 20090372
May 11, 2010 (FI) 20105517

(51) Int. Cl. A61K 31/215; A01N 37/08; A01N 25/10; A61P 31/04; A01P 1/00; C09D 193/00; A61P 31/10; C09D 5/16; C09D 5/14; C08L 23/12; C08L 63/00; C08L 21/00; A61K 9/00; A01P 15/00

(52) U.S. Cl. 424/405; 514/510; 424/400; 523/122; 106/203.3

(57) ABSTRACT

An antimicrobial composition containing coniferous resin acids and/or their derivates, an antimicrobial polymer composition including coniferous resin acids and/or their derivates and processes for preparing thereof, and the use of the derivates of coniferous resin acids as an antimicrobial agent.

### FIELD OF THE INVENTION

The invention relates to an antimicrobial composition comprising coniferous resin acids and/or their derivates. The invention also relates to an antimicrobial polymer composition comprising coniferous resin acids and/or their derivates. The invention further relates to the use of derivates of coniferous resin acids as an antimicrobial agent.

### BACKGROUND OF THE INVENTION

For centuries, tar has been used for protecting the bottom hull and deck structures of boats and ships. Later on, the growth of barnacles at the bottom of ships and boats was prevented by using antifouling paints made of substances containing heavy metals. At present, however, they are considered problematic for the environment, wherefore they are or will soon be forbidden. Barnacles at the bottom of boats and ships hinder the movement of ships and, thus, lead to an increase in costs and environmental load. The EU has also restricted the use of tar because of carcinogenic substances that are produced when tar is burnt and made.

Protection of various surfaces, such as walls, floors, counters, etc., against microbial growth, especially mould and barnacles, presents a problem, and attempts have been made to develop different protective agents and methods. There is a great demand for coatings, protective paints and materials that are as environmentally safe as possible. Previously, the protection was carried out, for instance, by different biocides, such as silver, tin, lead and copper compounds, quaternary polyatomic compounds, halides, such as different chlorine and bromic compounds, antibiotics, radiation, such as UV or radioactive radiation, and oxidization.

There is a wide variety of surface materials that need to be protected. Various objects and surfaces can be made of wood, plastic, steel, metal, rock, glass, ceramic materials, for example.

A widely used material is plastic. Plastic is used for constructing different surface structures that come into contact with the outside world and, thus, with micro-organisms and microbes. Plastic surfaces form a potential substrate for growth of micro-organisms and microbes. The growth of micro-organisms and microbes on plastic surfaces forms a potential reservoir (epidemiologic reservoir; microbial biofilm) and causes a risk that microbes move from one person to another in situations where many different people and animals touch or handle the same object or surface. A biofilm consisting of micro-organisms and microbes on the surface of different materials, such as plastic, may also destroy or damage the material surface. Therefore, hygiene and sterilization of plastic surfaces and planes are nowadays widely studied. This relates to spaces made of plastic in operating theatres, hospitals and the food industry, for example, and various coatings in spaces, such as floor coatings, counters, plastic items, etc.

A special surface to be protected with quite special safety demands for an antimicrobial composition is human or animal skin. It is obvious that substances injurious to health should be avoided in such compositions. However, there are products, such as hand disinfectants, on the market that contain, as an antimicrobial active agent, substances classified as skin-irritating or injurious to health.

A disadvantage of the prior art solutions is generally the great damage they cause to the environment. Antifouling paints contain heavy metals, which cause environmental load. Antibiotic agents have also been used to render surfaces and material antimicrobial, but this causes the problem of antibiotic-resistant strains. Surfaces and coatings have also been sterilized. A drawback of sterilization, in turn, is that the effect is only temporary, difficult to accomplish and that the process is expensive.

### BRIEF DESCRIPTION OF THE INVENTION

It has been found that coniferous resin acids possess antimicrobial properties against fungi. Surprisingly, also derivates of the coniferous resin acids have antimicrobial properties. The derivate can be, for example, an ester such as ethyl ester, isopropyl ester or glycerol ester of the resin acids. Further, it has been detected that the antimicrobial characteristics of the coniferous resin acids and their derivates are very extensive and are directed not only to bacteria but also very much to fungi and moulds.

Coniferous resins acids are advantageously available in rosin, such as in spruce resin, and tall oil fraction obtained as a by-product in kraft pulping process of wood. Rosin primarily includes abietic acid type (e.g. abietic acid, dehydro abietic acid) and pimaric acid type (e.g. pimaric acid, isopimaric acid) resin acids. Minor amounts of p-cumaric acid and lignans can also be included in the rosin.

An aspect of the invention is thus to provide an antimicrobial composition containing coniferous resin acids and/or their derivates. The composition may be, for instance, paint, wax, oil, washing agent, surface treatment agent, natural or synthetic fibre, or plastic article.

An aspect of the invention provides an antimicrobial polymer composition in which coniferous resin acids and/or their derivates are included into the matrix of the polymer composition. An embodiment of the invention provides an antimicrobial biomedical device made of polymeric material, where coniferous resin acids and/or their derivates are included in the polymeric material. In an embodiment, the biomedical device is a catheter, for example a urinary catheter.

The antimicrobial composition and the polymer composition of the invention provide the advantage that coniferous resin acids are derived from natural source, belonging to tree extractives, and can be obtained as wood industry by-products, which can now be further utilized. Moreover, glycerol ester of abietic acid, ‘Ester gum’, is classified among the substances allowable in food industry, having E number 445. Coniferous resin acids and their derivates provide very good long-term antimicrobial properties for the material treated with it. The compositions of the invention are thus healthy and environmentally friendly.

### BRIEF DESCRIPTION OF THE DRAWINGS

FIG. 1 shows a liquid-gas chromatogram (LGC) of Abicin® salve.

FIG. 2 shows a liquid-gas chromatogram (LGC) of Abicin® salve after hydrolysis of the salve.

FIG. 3 shows a liquid-gas chromatogram (LGC) of Abilar® salve.

### DETAILED DESCRIPTION OF THE INVENTION

According to an aspect, the invention provides an antimicrobial composition containing coniferous resin acids and/or their derivates.

In the present invention, the term “coniferous resin acids” is meant to include any raw material including various coniferous resin acids. Preferably, the raw material is derived from a natural source, like rosin such as spruce resin, and a fraction of resin acids obtained by distilling crude tall oil derived from kraft pulping process of wood, such as Taiga 2010 from Taiga Polymers. The term also includes any individual resin acid compound in its pure form, arbitrary mixtures of these compounds, and arbitrary mixtures of the raw materials including coniferous resin acid compounds.

A derivate of the coniferous resin acids can be produced in any conventional manner in the art. In an embodiment, the derivate is an ester of the resin acids. The ester can be, for example, ethyl ester, isopropyl ester or glycerol ester. An ester of the resin acids can be formed with any mono-, di- or polyhydric alcohol or any other ester-forming substance known in the art. For example, ethyl ester of the resin acids is obtained by reaction of the resin acids with ethanol. The esterification reaction is an equilibrium reaction where the relative amount of the resin acids and an ester can be controlled in a desired manner by reaction conditions. An ester formation is enhanced, for example, by removal of alcohol or water from the reaction mixture that are produced in the esterification reaction.

The composition of the invention is applicable to treatment of any material that is to be protected against microbial growth (bacteria, fungi, moulds, yeast, viruses). The material to be treated may be solid or in soluble form. The material may be, for instance, solution, solvent, wax, salve, paint, plastic, rubber, wood, rock, glass, ceramic material, paper, cardboard, paperboard, steel, metal, fibre, cloth, silicone, plaster. The material may also be human or animal skin. It may thus deal with washing or cleaning agents and disinfection agents, for example.

The composition may be mixed, spread, sprayed, injected, brushed, melted, absorbed, impregnated, dipped or otherwise brought to the material, which is rendered antimicrobial. Thus, the composition may be either spread onto the material surface or included in the material structure by, for example, mixing, absorption or melting, whereby the antimicrobial properties are provided homogenously along the entire material, instead of only in the surface of the material. This can be realized for different plastics in particular, the abietic acid raw material already being added to the raw materials of plastic during polymerization.

A raw material containing abietic acid, such as resin, may be added to the material to be protected as such, or it may first be dissolved in a suitable solvent. In many applications, the raw material is first dissolved suitably in an organic solvent, such as methanol, ethanol, isopropanol, acetone, ether, chloroform or formaldehyde, so as to have a concentration of 65% by weight, after which it is added to the mixture to be treated, such as wax, paint or cleaning agent. As a solution, the antimicrobial composition may also be absorbed into the object to be protected, such as wood. In water-insoluble paints or similar surface treatment solutions, absorption may be further boosted by the presence of commonly used carriers, such as linseed oil or paraffin oil.

The composition of the invention may also be spread onto the surface of the material, whereby a thin protective film, such as a resin film, is formed on the material after evaporation of the solvent. A resin film is highly water-insoluble and thus forms a highly water-insoluble protective film on the surface of the material. To boost the adhesion of the composition of the invention to the surface, the solution base should be a product having as good as possible adhesion properties with respect to the surface material to be protected. The treatment can be repeated, whereby the thickness of the layer on the surface to be protected can be adjusted. The thickness may also be adjusted by varying the concentration of the abietic acid raw material of the composition to be spread.

In an embodiment of the invention, spruce resin is blended with cellulose fibres which are modified by an enzyme. The modified fibres having antimicrobial properties are especially suitable for manufacturing of nonwoven products which can be used in hospital textile products, for example. By blending spruce resin with cellulose fibres before processing said fibres into a final product the antimicrobial properties are provided throughout into the fibre matrix of the product.

The amount of coniferous resin acids in the antimicrobial composition of the invention is typically from 0.1 to 20% (w/w or w/v). The amount may also be bigger but may then cause deterioration of physical properties, such as hardness, in the material to be protected. A suitable amount is determined according to the application of the composition. For example, boat waxes typically contain 0.5 to 10% by weight of abietic acid raw material.

According to another aspect, the invention provides an antimicrobial polymer composition containing coniferous resin acids. The polymer composition may be of thermosetting type or thermoplastic type plastic. Each plastic type is manufactured in its typical polymerization process and under its characteristic conditions. Coniferous resin acids may be added to the raw material mixture at the manufacturing stage of a polymer composition or included in the finished polymer composition homogenously by means of heat treatment, for instance. The most suitable process is selected according to the polymer type and the state of the coniferous resin acids raw material, for example. By adding coniferous resin acids, such as a resin solution, to a polymer composition already during formation of the composition, i.e. polymerization, it is possible to provide the polymer composition with antimicrobial properties throughout the entire thickness of the composition instead of only in the surface thereof.

Coniferous resin acids can be added to a polymer composition, for example, in amount of 0.1 to 15% (w/w or w/v), preferably approximately 10% (w/w or w/v). The amount of the resin acids to be added to a polymer composition is small enough not to affect the physical properties of the finished polymer composition but big enough to provide antimicrobial properties to the composition.

An embodiment of the invention is an epoxy having coniferous resin acids included throughout in the matrix of the epoxy. The epoxy is manufactured by adding coniferous resin acids to epoxide resin prior to subjecting it to curing, i.e. polymerization, with a hardener, typically polyamine. Alternatively, coniferous resin acids can be mixed with a hardener prior to adding it to epoxide resin for curing.

As stated above, the coniferous resin acids can also be impregnated into a polymer composition. The polymer composition can be, for example, a biomedical device, like a catheter, such as a urinary catheter, made of silicone or another rubber material, polyethylene, PVC or any other polymeric material suitable for biomedical devices.

Antimicrobial plastic composition can also be granulates which can be further subjected to extrusion or injection moulding in a conventional manner for preparing biomedical devices.

An antimicrobial catheter, for example, can be manufactured as follows. Coniferous resin acids, i.e. an antimicrobial active substance such as Taiga 2010, are dissolved in a solvent such as acetone, heptane, ethanol, isopropanol, styrene or the like. If desired, stabilizing agents and surfactants can be added. The solution or dispersion obtained is then brought into contact with the catheter by dipping spraying, tumbling or similar methods. Preferably, swelling of the catheter with an active substance is accomplished in a closed system, such as in a CO₂ reactor which is later used for selective removal of the solvent.

Although the swelling ability of a catheter in a solvent depends on the polymeric material from which the catheter is made of, some solvent and hence, also some of the active substance will be impregnated into the catheter. The absorbed amount of an active substance can be controlled by adjusting the concentration of the active substance in a solvent, the exposure time of the solvent including an active substance and duration of subsequent CO₂ treatment.

For impregnation, the catheter is placed in a reactor which can be evacuated and pressurized. After evacuation and/or replacement of gas volume by inert gas such as CO₂, the solvent containing an active substance is sprayed over the catheter. For example, catheter made of silicone absorbs a sufficient amount of Taiga 2010 in 5 minutes. After impregnation, excess solution is removed, for example by heating or sonication.

Pressurized CO₂ is added for dissolving residual solvent. Appropriate pressure is above 20 bar, preferably above 30 bar, more preferably above 40 bar. Suitable temperature is an ambient temperature, for example about 10° C. to about 15° C. CO₂ can be applied more than once, for example by using two or more baths to remove the solvent as much as possible. Liquid CO₂ or supercritical CO₂, preferably liquid CO₂ is used in the invention. CO₂ is finally washed out from the catheter.

The antimicrobial polymer composition of the invention may further be used as a raw material for manufacturing plastic products. Thus, plastic products made of an antimicrobial polymer composition, such as plastic cutlery, toys, etc., are antimicrobial as such and do not require a separate treatment for disinfecting or sterilizing the product.

The antimicrobial composition of the invention may be added to thermosetting plastics, such as polyester, epoxies, polyurethane and phenolic plastics. The composition of the invention may further be added to inorganic thermosetting plastics, such as silicic acid polymers, e.g. silicone. These may be used in the production of sanitary silicones or silicone catheters, for instance.

The composition of the invention may also be added to organic plastics, such as polymers based on plants and animals or oil-based synthetic plastics. These can be used for manufacturing, for example, floor, wall and roof coatings, damp-proofing, sauna and bathroom sprays, bottles, cooking utensils, displays, printed circuit boards, glassy carbon and aramid composite materials, boats, conductors, insulation plates and foams, foam plastic, wheels and cables, glues and sealing materials.

The antimicrobial composition of the invention may also be added to thermoplastics, such as acryl, polyethylene, polypropylene, polystyrene, polyethylene terephthalate, polyvinyl chloride, polyamide, polycarbonate, polytetrafluoroethylene, polyoxymethyl or polyacetal, ethylenechiorofluoroethylene, polyvinyl fluoride, and acrylonitrilebutadienestyrene. These may be used for manufacturing, for instance, paints, coatings, decoration, glass substitutes, brushes, plastic bags, pouches, ropes, household utensils, car parts, textiles, pipes, cooking utensils, toys, bottles, conductors, insulators, lenses, various coatings (Teflon) and seals, coatings for containers and piping of the processing industry, filters, valves, bearings, clothes and accessories, cloths, domestic appliances and office equipment, boats, boxes, barnacles and switches, plugs, plastic padding, plastic cards, keyboards, bars and hangers, glues and seaming materials. By way of example, an antimicrobial floor carpet made of polypropylene may be mentioned.

The composition of the invention may be added to fibres, such as animal, plant, mineral and synthetic fibres, e.g. thread, pulp, paper, carbon fibre (composite materials), viscose fibre, nylon, polyester, Kevlar, elastane and polyester. These may be used for manufacturing air filters, fabrics, clothes and accessories, packaging materials, glass fibre boats, wallpapers and mortars or banknotes, for example. Antimicrobial viscose fibre may be used for manufacturing of nonwoven products for various hospital textiles used in operating rooms, for instance.

The composition of the invention may be added to rubber, both natural rubber and synthetic rubber, and thermoplastic elastomers. These can be used for manufacturing switches, connection pieces, hoses, seals, plugs, sealing materials, shoes, clothes and accessories, condoms and toys.

The composition of the invention may be added to glass, from which coated glasses, lenses and bottles can be made.

The composition of the invention may be added to chemicals, such as washing and cleaning agents and disinfectants. They can be used for manufacturing anti-scurf shampoos, cleaning agents and disinfectants, for example.

The composition of the invention may be added to emulsions and salves. They can be used for manufacturing products for treating wounds and nail fungus.

As mentioned above, the composition of the invention may also be added to wood and metal.

A material treated with the antimicrobial composition of the invention and the antimicrobial polymer composition of the invention may be used in many different applications. Examples of such applications include medical and veterinary applications. Examples of these include plastic cloths for protection of surgical sites, such as skin, and injuries and coatings (protective film) to be spread onto the skin or tissues, coatings for suture thread, hooks and clamps, and similar plastic products, protective gloves, hair covers, aprons, etc., as well as coatings thereof, plasters, wound dressings, rinsing liquids (for cavities like abdominal cavity, outer auditory canal, sinus, oral cavity, etc.), plastic catheters (such as urinary catheters, drains, etc.), hoses, cannulae and coatings thereof. Examples of medical and veterinary applications also include plastic protective cloths and bandages for surgical wounds and skin openings made during operations (e.g. colostomy, ileostomy, etc.), artificial plastic parts for the body or body cavities (prostheses and artificial transplants, oral prostheses, dental bridges, implants, tubes, nails, etc.) and coatings thereof. Examples of medical and veterinary applications also include coatings for artificial parts to be placed into the body and made of metal or material other than plastic. Examples of applications also include plasters, splints, supporting prostheses, protective bandages, shoes, insoles and coatings thereof.

In addition, the antimicrobial compositions of the invention may be used in operating rooms, hospitals, showers and nursing equipment for domestic use, such as instruments, cups, bowls, furniture, e.g. counters, tables, handles, air conditioning ducts and coatings thereof.

The invention also relates to the use of abietic acid raw material as an antimicrobial agent.

The following examples illustrate the invention.

### EXAMPLES

Example 1 illustrates the antimicrobial properties of an abietic acid ester.

In Examples 2 to 8, the microbicidal effect of the antimicrobial compositions of the invention was measured according to the standard EN 13697, which relates to a quantitative surface test for the evaluation of bactericidal and/or fungicidal activity of chemical disinfectants used in food, industrial, domestic and institutional areas. The tested bacterial, fungicidal and mould strains were in accordance with the standard. From each test organism, colonies forming units (cfu) of the test suspension per ml (N) and the microbicidal effect (Nₐ) of the test substance are determined. According to the standard, a good microbicidal effect is achieved when microbial reduction for bacteria is log4 and for fungi log3.

### Example 1

FIG. 1 illustrates a liquid-gas-chromatogram (LGC) of an acetone extract of the native Abicin® laquer including coniferous resin acids and monopropylene glycol. It is known that Abicin® laquer has antimicrobial properties.

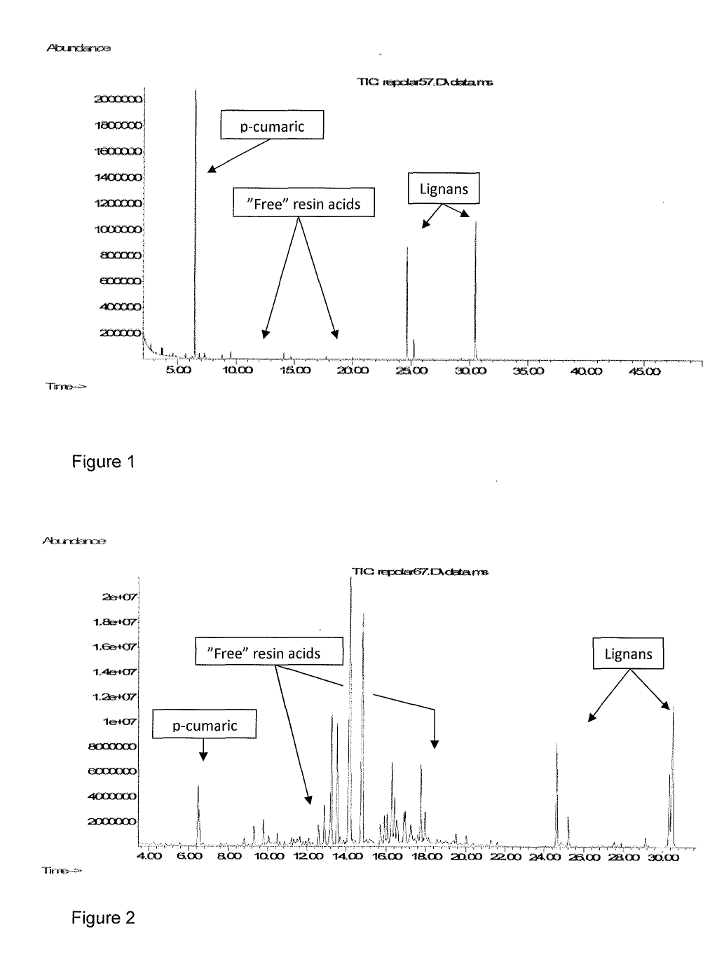

FIG. 2 illustrates a liquid-gas-chromatogram (LGC) of an acetone extract of Abicin® laquer after hydrolysis of the laquer.

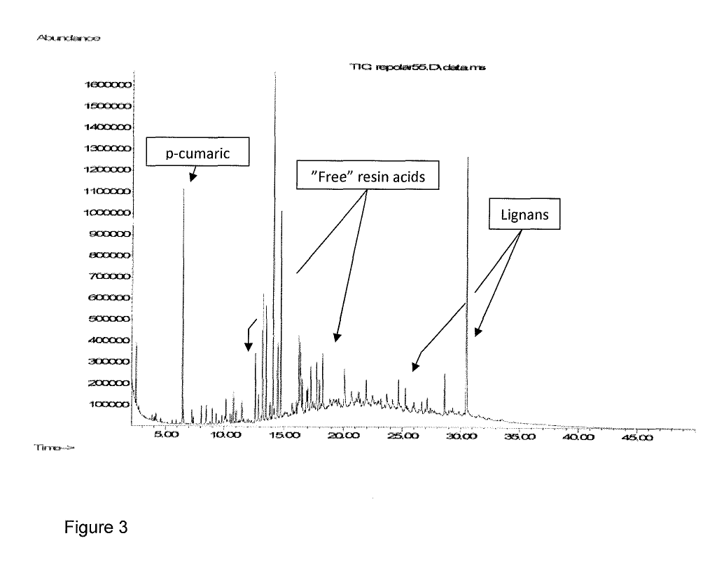

FIG. 3 illustrates a liquid-gas-chromatogram (LGC) of an acetone extract of the native Abilar® salve including coniferous resin acids and no alcohol.

It can be seen that the spectrum of FIG. 2 is similar to that of FIG. 3. The resin acids do not appear in the chromatogram of FIG. 1 but will appear in that of FIG. 2. Based on the spectra of FIGS. 1 to 3, it can be concluded that hydrolysis of Abicin® laquer results in free resin acids and alcohol. The resin acids in the native Abicin® laquer having antimicrobial properties are thus present in an esterified form.

### Example 2

The microbicidal effect of a one-percent A12t alcohol solution made of spruce resin, a control solution and a reference solution was tested on a steel test plate. The effective time of the samples on the plate was 24 hours. The results are given in Table 1.

The meanings of the sample numbers are as follows:
0=A12t alcohol (control)
1=one-percent spruce resin solution in A12t alcohol (according to the invention)
2=Erisan® surgical hand disinfectant (reference)
3=clean steel plate (test plate).

Erisan® hand disinfectant contains hexadecyl trimethyl ammonium chloride, which is classified as irritating to skin and hazardous to health if swallowed.

TABLE 1

| Sample/ | | Ent. | Ps. | | | |
|---|---|---|---|---|---|---|
| microbe | S. aureus | hirae | aeruginosa | E. coli | A. niger | C. albicans |
| 0 | log 1 | log 1 | log 1 | log 1 | log 1 | log 1 |
| 1 | >log 3 | >log 3 | >log 3 | >log 3 | >log 3 | >log 3 |
| 2 | >log 3 | log 1 | >log 3 | >log 3 | >log 3 | >log 3 |
| 3 | log 1 | log 1 | log 1 | log 1 | log 1 | log 1 |

The results show that denaturated alcohol does not provide long-term protection against microbes. The reference sample gives good protection against all bacteria but not the enterococcus bacterium. The composition of the invention provides good long-term protection against all microbes tested.

### Example 3

A 0.5-percent spruce resin solution was produced into an A12t alcohol solution. The microbicidal effect of the obtained solution was tested after an effective time of 24 hours. The results are shown in Table 2.

TABLE 2

| | cfu/ml | | |
|---|---|---|---|
| | at the beginning | | |
| Microbe | of the test (N) | after the test (Nₐ) | bacterial reduction |
| S. aureus | 3.31 × 10⁸ | 1.10 × 10⁴ | 2.31 × 10⁴ |
| E. coli | 1.94 × 10⁸ | 1.00 × 10² | >10⁵ |
| Ps. aeruginosa | 4.11 × 10⁸ | 1.00 × 10² | >10⁵ |
| Ent. hirae | 2.74 × 10⁸ | 3.74 × 10⁴ | 2.34 × 10³ |
| C. albicans | 2.07 × 10⁸ | 3.48 × 10⁴ | 1.77 × 10³ |
| A. niger | 1.12 × 10⁷ | >10⁵ | <10² |

The results show that the resin-containing solution gives good long-term protection against bacterial and fungicidal strains and also clearly prevents the growth of mould (A. niger).

### Example 4

A one-percent solution of Taiga 2010 was produced into an A12t alcohol solution. The microbicidal effect of the obtained solution was tested after an effective time of 5 minutes. The results are shown in Table 3.

TABLE 3

| | Cfu/ml | | |
|---|---|---|---|
| | at the beginning | | |
| Microbe | of the test (N) | after the test (Nₐ) | bacterial reduction |
| S. aureus | 1.86 × 10⁸ | <5 × 10² | >10⁵ |
| E. coli | 1.22 × 10⁸ | <5 × 10² | >10⁵ |
| Ps. aeruginosa | 1.32 × 10⁸ | <5 × 10² | >10⁵ |
| Ent. hirae | 2.03 × 10⁸ | <5 × 10² | >10⁵ |
| C. albicans | 4.1 × 10⁷ | <5 × 10² | >10⁵ |
| A. niger | 4.8 × 10⁷ | <5 × 10² | >10⁵ |

The results show that the solution of the invention provides a good microbicidal effect.

### Example 5

A 0.5-percent solution of Taiga 2010 was produced into an A12t alcohol solution. The microbicidal effect of the obtained solution was tested after an effective time of 24 hours. The results are shown in Table 4.

TABLE 4

| | Cfu/ml | | |
|---|---|---|---|
| | at the beginning | | |
| Microbe | of the test (N) | after the test (Nₐ) | bacterial reduction |
| S. aureus | 3.31 × 10⁸ | 1.72 × 10⁴ | 1.59 × 10⁴ |
| E. coli | 1.94 × 10⁸ | <5 × 10² | >10⁵ |
| Ps. aeruginosa | 4.11 × 10⁸ | 1.3 × 10² | >10⁵ |
| Ent. hirae | 2.74 × 10⁸ | <5 × 10² | >10⁵ |
| C. albicans | 2.07 × 10⁸ | 6.78 × 10⁴ | 1.39 × 10³ |
| A. niger | 1.12 × 10⁷ | >10⁵ | <10² |

The results show that Taiga 2010 gives good long-term protection against bacterial and fungicidal strains and also clearly prevents the growth of mould (A. niger).

### Example 6

The microbicidal effect of an epoxy resin, in which 10% by weight of Taiga 2010 was included into the matrix of the epoxy resin, was tested. A standardized amount (N) of microbes was pipetted onto the surface of a hardened plastic plate in a culture liquid, after which the plate was incubated for 24 hours. After this, a sample (Nₐ) was taken from the test area, and the sample was grown on a specific substrate for growth for 24 hours. Bacterial reduction was calculated from the difference N-Nₐ. The results are shown in Table 5.

TABLE 5

| | Cfu/ml | | |
|---|---|---|---|
| | at the beginning | | |
| Microbe | of the test (N) | after the test (Nₐ) | bacterial reduction |
| S. aureus | 1.86 × 10⁸ | 1.16 × 10⁸ | 7.0 × 10 |
| E. coli | 1.22 × 10⁸ | <5 × 10² | >10⁵ |
| Ps. aeruginosa | 1.32 × 10⁸ | 4 × 10² | >10⁵ |
| Ent. hirae | 2.03 × 10⁸ | 6.28 × 10⁴ | 1.9 × 10³ |
| C. albicans | 4.1 × 10⁷ | 5.0 × 10³ | 3.6 × 10³ |
| A. niger | 4.8 × 10⁷ | 1.4 × 10⁴ | 3.4 × 10³ |

The results show that a good long-term microbicidal effect is achieved with Taiga 2010 included in the epoxy resin.

### Example 7

The microbicidal effect of a polyester polymer, in which 10% by weight of Taiga 2010 was included in the polymer matrix, was tested. Taiga 2010 was dissolved in styrene which was used in a component in a polymerization reaction of the polyester polymer. The effect was measured after Taiga 2010 had been effective in the polymer for 24 hours. The test was performed like in Example 5.

TABLE 6

| | Cfu/ml | | |
|---|---|---|---|
| | at the beginning | | |
| Microbe | of the test (N) | after the test (Nₐ) | bacterial reduction |
| S. aureus | 1.50 × 10⁸ | <5 × 10² | >10⁵ |
| E. coli | 1.10 × 10⁸ | <5 × 10² | >10⁵ |
| Ps. aeruginosa | 1.42 × 10⁸ | <5 × 10² | >10⁵ |
| Ent. hirae | 1.04 × 10⁸ | <5 × 10² | >10⁵ |
| C. albicans | 4.7 × 10⁷ | <5 × 10² | >10⁵ |
| A. niger | 3.5 × 10⁷ | <5 × 10² | >10⁵ |

The results show that a good long-term microbicidal effect is achieved with Taiga 2010 included in the polyester polymer.

### Example 8

Epoxy was prepared in a conventional manner from epoxide resin and a hardener. Coniferous resin acids (5% w/w) were mixed with an epoxide resin. A hardener is added to a mixture obtained to initiate a polymerization reaction. Hardened epoxy resin is achieved having coniferous resin acids homogenously divided throughout in its matrix structure.

Antimicrobial characteristics of the epoxy resin are shown in Table 7 below. Growth of various microorganisms on the surface of the epoxy resin was analyzed. A standardized amount (N) of microbes (1 to 5×10⁵/test bacteria and 1 to 5×10⁴ pmy/test yeast/mould/fungi) was pipetted onto the surface of the epoxy resin in a culture liquid, after which the contact plates were incubated for 24 hours.

TABLE 7

| Plate | MRSA | Ent. hirae | Ps. aeruginosa | E. coli | A. niger | T. rubrum | C. albicans |
|---|---|---|---|---|---|---|---|
| Resin (2% | − | − | − | − | + | − | − |
| w/w) + epoxy | | | | | | | |
| Resin (10% | − | − | − | − | + | − | − |
| w/w) + epoxy | | | | | | | |
| Epoxy | ++ | ++ | − | + | ++ | + | ++ |
| Lab. Ref. | ++ | ++ | − | + | ++ | + | ++ |

Abbreviations:
−no growth;
+mild growth;
++strong growth of microbes

It can be seen from the results that epoxy resin including coniferous resin acids significantly reduces or eliminates the growth of various microbes on a surface thereof.

### Example 9

The example shows that a thin film made of spruce resin acids and provided on the plastic substrate prevents the growth of microorganisms, i.e. sea barnacles, considerably.

Two 26×20 cm plastic plates made of polyurethane were coated with a resin film so that 10% by weight of purified spruce resin solution in 70-percent alcohol was spilled as a thin layer onto the plastic plate, after which the plate was allowed to dry (alcohol and water evaporated). Thus, a thin solid “resin film” is formed on the plastic plate. Two similar plates were left uncoated, these being the back sides of each coated plate. The plates were immersed in sea water into a depth of 1 metre for 3 months. Morphometrically calculated (P<0.001), the growth of sea barnacles on the plates coated with a resin film (the number of barnacles on the surface of the entire plate) was 22% when compared to the number of barnacles on reference plates without a resin film. The barnacles attached to the control plate were also smaller than the barnacles on the plates treated with a resin solution. The results are shown in Table 8.

TABLE 8

| | | | | | | Mean | Standard |
|---|---|---|---|---|---|---|---|
| Resin plates | Number of barnacles | | | | | value | deviation |
| 1. resin plate | 83 | 71 | 88 | 80 | 82 | 81 | 6.2 |
| 2. resin plate | 12 | 12 | 13 | 13 | 10 | 12 | 1.2 |
| Reference plates | | | | | | | |
| 1. reference plate | 322 | 387 | 390 | 370 | 401 | 374 | 31.1 |
| 2. reference plate | 136 | 125 | 159 | 150 | 142 | 142 | 13.0 |

The results show that a resin film prevents 80% of the attachment of barnacles and the growth thereof onto the plastic substrate.

### Example 10

Spruce resin was dissolved in an alkaline solution of cellulose fibres which were modified enzymatically. Resin dissolved easily in an alkaline solution without altering the properties of the cellulose solution. The added amount of spruce resin was 1% of dry matter content of cellulose fibres. In order to test the antimicrobial properties of the fibres treated with spruce resin, a film were precipitated from the cellulose solution by immersing a glass plate applied with the cellulose solution into a 15% sulphuric acid solution. A reference film was prepared in a similar manner from modified viscose fibres without adding spruce resin thereto. Antimicrobial test of the film was performed according to ISO standard 20645/20743.

The antimicrobial test results showed that the film made of the viscose fibres of the invention showed excellent microbicidal properties (<10⁵) against S. aureus, E. coli and K. pneumoniae while the reference film exhibited no antmicrobial properties.

1. An antimicrobial composition containing coniferous resin acids and/or their derivates.

2. The composition of claim 1, wherein coniferous resin acids are derived from rosin.

3. The composition of claim 1, wherein coniferous resin acids are derived from crude tall oil obtained by kraft pulping of wood.

4. The composition of any of claims 1 to 3, wherein the derivate of coniferous resin acids is an ester.

5. The composition of claim 4, wherein the derivate is an ester of abietic acid.

6. The composition of claim 4, wherein the derivate is an ester of dehydro abietic acid.

7. The composition of claim 4, wherein the derivate is an ester of pimaric acid.

8. The composition of any of the preceding claims, wherein coniferous resin acids are dissolved in an alcohol.

9. The composition of any one of the preceding claims, wherein the amount of coniferous resin acids and/or their derivates is 0.1 to 20% w/w or w/v of the composition.

10. The composition of any one of the preceding claims, which is a surface protection composition, such as a surface cleaning agent or hand disinfectant, paint, wax, varnish, oil, natural or synthetic fibre, fabric.

11. The composition of claim 10, wherein the natural fibre is a viscose fibre.

12. The composition of claim 11, wherein the viscose fibres contain about 1% w/w of spruce resin on dry matter basis of the viscose fibres.

13. The composition of claim 10, which is a nonwoven fabric made of viscose fibres defined in claim 12.

14. The composition of claim 10, which is a surface cleaning agent.

15. The composition of claim 10, which is a hand disinfectant.

16. An antimicrobial polymer composition comprising coniferous resin acids and/or their derivates in the matrix of the polymer composition.

17. The polymer composition of claim 16, wherein the amount of coniferous resin acids and/or their derivates is 0.1 to 20% w/w or w/v of the composition.

18. The polymer composition of claim 16 or 17, which is polypropylene fibre.

19. The polymer composition of claim 16 or 17, which is epoxy resin.

20. The polymer composition of claim 16 or 17, which is a biomedical device, such as catheter.

21. A process for manufacturing a polymer composition of any of claim 16 or 20, comprising addition of coniferous resin acids and/or their derivates to one of more monomers prior to initiation of a polymerization reaction of the monomers.

22. A process for manufacturing a polymer composition of any of claim 16 or 20, comprising impregnation of coniferous resin acids and/or their derivates in a solution into a polymer composition matrix for a sufficient time to render the polymer composition antimicrobial.

23. The process of claim 22, wherein the impregnation is performed by using supercritical CO₂.

24. A use of derivates of coniferous resin acids as an antimicrobial agent.

25. The use of claim 24, wherein the derivate is an ester.

26. The use of claim 24 or 25 in
plastic cloths for protection of surgical sites, such as skin, and injuries,
coatings to be spread onto the skin or tissues,
coatings for suture thread, hooks and clamps,
protective gloves, hair covers, aprons as well as coatings thereof,
plasters, wound dressings, rinsing liquids for cavities like abdominal cavity, outer auditory canal, sinus, oral cavity,
plastic catheters such as urinary catheters and drains, hoses, cannulae and coatings thereof,
bandages for surgical wounds and skin openings made during operations, e.g. colostomy, ileostomy,
artificial plastic parts for the body or body cavities like prostheses and artificial transplants, oral prostheses, dental bridges, implants, tubes, nails, and coatings thereof,
coatings for artificial parts to be placed into the body and made of metal or material other than plastic, plasters, splints, supporting prostheses, protective bandages, shoes, insoles and coatings thereof.
